(19) United States
(12) Patent Application Publication
(10) Pub. No.: US 2024/0359035 A1
(43) Pub. Date: Oct. 31, 2024

(54) PARTICLE BEAM CONTROL SYSTEM AND PARTICLE BEAM CONTROL METHOD

(71) Applicants: KABUSHIKI KAISHA TOSHIBA, Tokyo (JP); TOSHIBA ENERGY SYSTEMS & SOLUTIONS CORPORATION, Kawasaki-shi (JP)

(72) Inventors: Atsuri MIYAUCHI, Mitaka Tokyo (JP); Nobukazu KAKUTANI, Yokohama Kanagawa (JP); Katsushi HANAWA, Kita Tokyo (JP); Shinya FUKUSHIMA, Fuchu Tokyo (JP)

(73) Assignees: KABUSHIKI KAISHA TOSHIBA, Tokyo (JP); TOSHIBA ENERGY SYSTEMS & SOLUTIONS CORPORATION, Kawasaki-shi (JP)

(21) Appl. No.: 18/766,952

(22) Filed: Jul. 9, 2024

Related U.S. Application Data
(63) Continuation of application No. PCT/JP2023/018913, filed on May 22, 2023.

(30) Foreign Application Priority Data
Jun. 8, 2022 (JP) 2022-092732
May 19, 2023 (JP) 2023-082839

(51) Int. Cl. A61N 5/10; G21K 1/093

(52) U.S. Cl. CPC A61N 5/1065; A61N 5/1049; A61N 5/1077; G21K 1/093; A61N 2005/1087

(57) ABSTRACT

According to one embodiment, a particle beam control system comprising: a control computer configured to control two scanning electromagnets, wherein the control computer is configured to: calculate deviation amount between a centroid position and a spot position that is a designed irradiation position of the two scanning electromagnets; calculate at least one correction value for correcting the centroid position to the spot position by using the deviation amount; store the at least one correction value in a memory; and correct at least one current value by using the at least one correction value stored in the memory, the at least one current value being a design reference when power is supplied from at least one of two power supplies to at least one of the two scanning electromagnets.

### CROSS-REFERENCE TO RELATED APPLICATIONS

This application is a Continuation Application of No. PCT/JP2023/018913, filed on May 22, 2023, and the PCT application is based upon and claims the benefit of priority from Japanese Patent Applications No. 2022-092732, and No. 2023-082839, filed on Jun. 8, 2022, and May 19, 2023, the entire contents of which are incorporated herein by reference.

### TECHNICAL FIELD

Embodiments of the present invention relate to techniques for particle beam control.

### BACKGROUND

In a particle beam treatment apparatus, a scanning irradiation method is known as a method in which an irradiation field in a cross-section perpendicular to the axis of the particle beam is filled with the particle beam. In this method, a lesion site can be irradiated with the particle beam three-dimensionally and precisely without using a collimator or a bolus. For example, when the particle beam is deflected in two directions by using two scanning electromagnets that generate magnetic fields in different directions, the particle beam can be radiated two-dimensionally. When these scanning electromagnets configured to be different in magnetic field direction from each other are integrated, its advantage is that the magnetic field can be generated efficiently and the distance from the scanning electromagnet to the irradiation position can be shortened. However, it is difficult to precisely position the scanning electromagnet such that the magnetic field in the horizontal direction and the magnetic field in the vertical direction are exactly orthogonal to each other.

When these magnetic fields in the horizontal and vertical directions are exactly orthogonal to each other, the irradiation target surface can be accurately irradiated with the particle beam. For example, the particle beam can be radiated in such a manner that the irradiation field forms a precise square. However, when these magnetic fields in the horizontal and vertical directions are not exactly orthogonal to each other, the particle beam is diagonally deflected so as to be rotated or distorted with respect to the irradiation target surface. Furthermore, the magnetic field of the scanning electromagnet is distorted near the magnetic pole, and thus, the particle beam passing through the region near the magnetic pole cannot be radiated to an accurate location.

After positioning (i.e., alignment) of the particle beam, if it is found that the two magnetic fields in the horizontal and vertical directions are not orthogonal to each other, it is difficult to disassemble the scanning electromagnets and perform correction and reassembly. For example, positioning of the scanning electromagnets requires removal of peripheral devices, and thus, it is extremely time-consuming. Although rotation of the irradiation target surface of the particle beam can be corrected by rotating the entire scanning electromagnets around the axis, the only way to correct distortion of the irradiation target surface of the particle beam is to remanufacture the scanning electromagnets, and it takes a considerable amount of effort and time to eliminate this distortion as much as possible. For this reason, it is necessary to correct the scanning electromagnets in such a manner that the particle beam is radiated as required by the design without disassembling the scanning electromagnets. In particular, there is a demand to minimize the amount of work required for adjusting the deviation that is inevitably caused in the irradiation position of the particle beam in terms of manufacturing accuracy and installation accuracy.

### PRIOR ART DOCUMENT

### Patent Documents

[Patent Document 1] Japanese Patent No. 6602732
[Patent Document 2] JP 2014-103974 A
[Patent Document 3] Japanese Patent No. 6613466

### SUMMARY

### Problem to be Solved by Invention

The present invention aims to provide a particle beam control technique that can simplify the work of adjusting the deviation in the irradiation position of the particle beam.

### BRIEF DESCRIPTION OF DRAWINGS

FIG. 1 is a configuration diagram illustrating a particle beam control system.

FIG. 2 is a block diagram illustrating a control computer.

FIG. 3 is a flowchart illustrating correction processing of the particle beam.

FIG. 4 is a flowchart illustrating irradiation start processing of the particle beam.

FIG. 5 is a cross-sectional view illustrating an electromagnetic structure of the first modification.

FIG. 6 is a plan view illustrating the electromagnetic structure of the first modification.

FIG. 7 is a side view illustrating the electromagnetic structure of the first modification.

FIG. 8 is a side view illustrating the electromagnetic structure of the second modification.

### DETAILED DESCRIPTION

In one embodiment of the present invention, a particle beam control system comprising: two scanning electromagnets configured to scan a particle beam in two-dimensional directions and be different in direction of deflecting the particle beam from each other; two power supplies configured to supply respective powers to the two scanning electromagnets; a position monitor configured to detect a position of the particle beam; and a control computer configured to control the two scanning electromagnets, wherein the control computer is configured to: calculate a centroid position by using the position of the particle beam detected by the position monitor, the centroid position being an actual irradiation position of the particle beam; calculate deviation amount between the centroid position and a spot position that is a designed irradiation position of the two scanning electromagnets; calculate at least one correction value for correcting the centroid position to the spot position by using the deviation amount; store the at least one correction value in a memory; and correct at least one current value by using the at least one correction value stored in the memory, the at least one current value being a design reference when power is supplied from at least one of the two power supplies to at least one of the two scanning electromagnets.

According to embodiments of the present invention, it is possible to provide a particle beam control technique that can simplify the work of adjusting the deviation in the irradiation position of the particle beam.

Hereinbelow, embodiments of a particle beam control system and a particle beam control method will be described in detail by referring to the accompanying drawings.

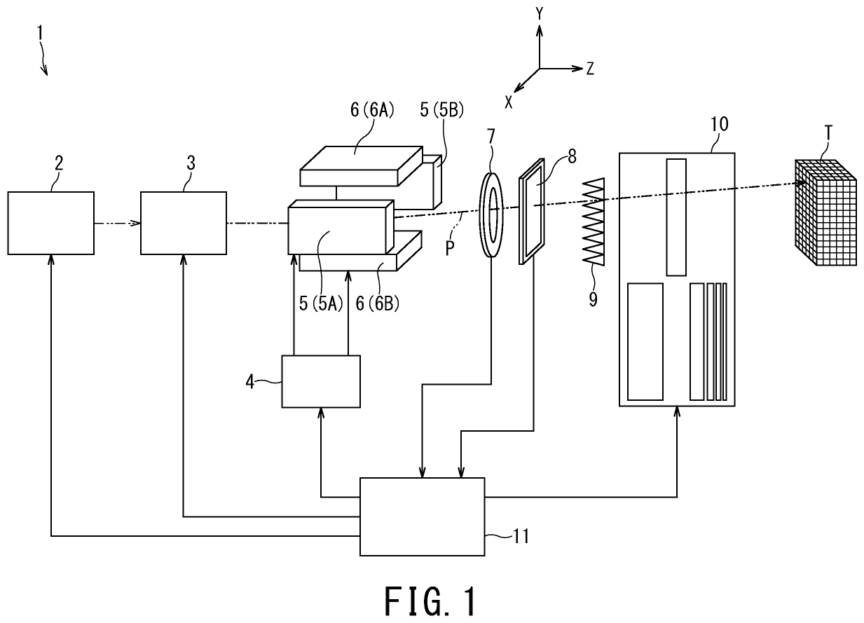

The reference sign 1 in FIG. 1 denotes a particle beam control system according to the present embodiment. The particle beam control system 1 is a so-called particle beam treatment apparatus that highly accelerates charged particles such as carbon ions and protons and performs treatment by irradiating a patient's lesion site such as a cancerous tissue with a controlled particle beam P.

A radiation therapy with the use of the particle beam P like this is also referred to as a heavy ion beam cancer treatment. This treatment is said to be able to damage the cancerous lesion (i.e., focus of disease) and minimize the damage to normal cells by pinpointing the cancerous lesion with carbon ions. Note that the particle beam is defined as radioactive rays heavier than an electron, and include a proton beam and a heavy ion beam, for example. Of this, the heavy ion beam is defined as radioactive rays heavier than a helium atom.

As compared with the conventional cancer treatment using X-rays, gamma rays, or proton beams, the cancer treatment using the heavy ion beam has characteristics that: (i) the ability to kill the cancerous lesion is higher; and (ii) the radiation dose is weak on the surface of the body of the patient so as to peak at the cancerous lesion. Thus, the number of irradiations and side effects can be reduced, and the treatment period can be shortened.

The particle beam P loses its kinetic energy at the time of passing through the body of the patient so as to decrease its velocity and receive a resistance that is approximately inversely proportional to the square of the velocity and stops rapidly when it decreases to a certain velocity. The stopping point of the particle beam P is referred to as the Bragg peak at which high energy is emitted. This Bragg peak is matched with the position of the lesion tissue of the patient, and thus, can kill only the lesion tissue while suppressing the damage to normal tissues.

In particular, a description will be given of embodiments of using the scanning irradiation method in which a lesion site T as the irradiation target is virtually divided into three-dimensional lattice shapes (i.e., lattice points) and three-dimensional scanning is performed in the X-axis direction (i.e., the horizontal direction), the Y-axis direction (i.e., the vertical direction), and the Z-axis direction (i.e., the depth direction).

In the following description, the actual irradiation position of the particle beam P on the lesion site T is referred to as “the centroid position”, and the designed irradiation position of the particle beam P in the particle beam control system 1 is referred to as “the spot position”. During construction or periodic maintenance of the particle beam control system 1, the centroid position is adjusted to match the spot position.

As shown in FIG. 1, the particle beam control system 1 includes a beam generator 2, a beam accelerator 3, a beam scanner 4, scanning electromagnets 5 and 6, a dose monitor 7, a position monitor 8, a ridge filter 9, a range shifter 10, and a control computer 11.

The beam generator 2 generates charged particles such as carbon ions and protons.

The beam accelerator 3 accelerates the charged particles generated by the beam generator 2 with a predetermined accelerator. The charged particles are accelerated by the beam accelerator 3 until they have enough energy to reach deep into the lesion site T, and then proceed as the particle beam P. The beam accelerator 3 controls on/off of emission of the particle beam P on the basis of a control signal outputted from the control computer 11.

The beam scanner 4 supplies powers to the scanning electromagnets 5 and 6 so as to control these scanning electromagnets 5 and 6. When the traveling direction of the particle beam P is defined as the Z-axis direction, the scanning electromagnets 5 and 6 can deflect the particle beam P in the X-axis direction and in the Y-axis direction. In other words, the scanning electromagnets 5 and 6 scan the particle beam P two-dimensionally over the slice surface of the lesion site T.

The configuration of the present embodiment includes two scanning electromagnets 5 and 6 that scan the particle beam P in two-dimensional directions and are different in the direction of deflecting the particle beam P from each other. One scanning electromagnet 5 or 6 is composed of a pair of two electromagnets (deflection coils), and the particle beam P passes between the pair of electromagnets.

For example, the scanning electromagnet 5 is composed of a pair of X-axis electromagnets 5A and 5B that deflect the particle beam P in the X-axis direction, and the scanning electromagnet 6 is composed of a pair of Y-axis electromagnets 6A and 6B that deflect the particle beam P in the Y-axis direction. The X-axis electromagnets 5A and 5B and the Y-axis electromagnets 6A and 6B are installed at the same position in the Z-axis direction, which is the traveling direction of the particle beam P. In this manner, the scanning electromagnets 5 and 6 can be made smaller in size. For example, as compared with the conventional case where the X-axis electromagnets 5A and 5B and the Y-axis electromagnets 6A and 6B are arranged in the Z-axis direction, the scanning electromagnets 5 and 6 of the present embodiment can be smaller in size in the Z-axis direction. However, the X-axis electromagnets 5A and 5B and the Y-axis electromagnets 6A and 6B are integrated, and thus, it is not easy to perform maintenance in which the scanning electromagnets 5 and 6 are disassembled. It also becomes difficult to finely adjust the relative positions of the X-axis electromagnets 5A and 5B and the Y-axis electromagnets 6A and 6B.

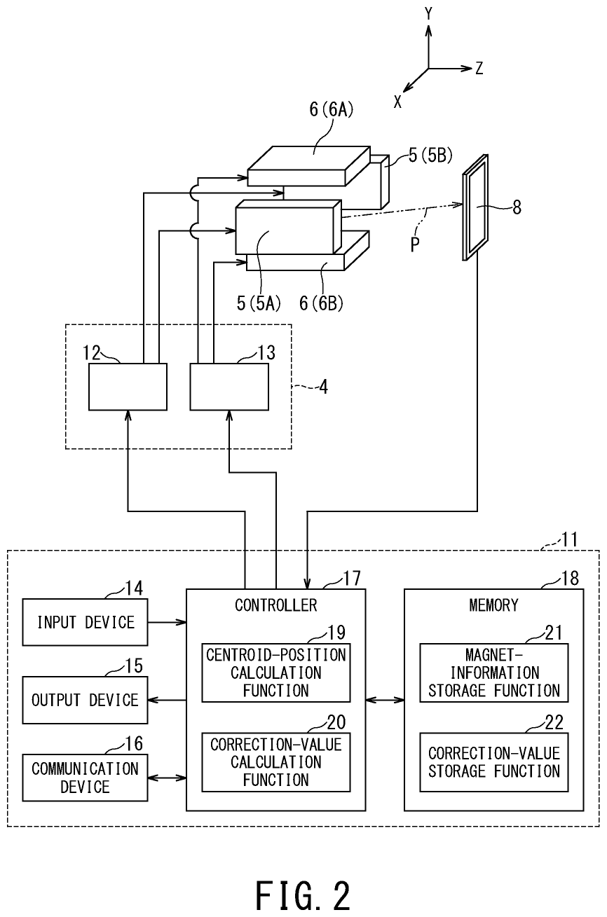

The beam scanner 4 controls respective excitation currents of the powers to be supplied to the X-axis electromagnets 5A and 5B and the Y-axis electromagnets 6A and 6B (FIG. 2).

The dose monitor 7 monitors the dose delivered to the lesion site T of the patient that is the irradiation target. The information indicating the dose of the particle beam P detected by the dose monitor 7 is inputted to the control computer 11.

The position monitor 8 detects the position of the particle beam P in the X-axis direction and in the Y-axis direction. For example, the position monitor 8 detects the position of the particle beam P scanned during particle beam treatment, and detects whether there is any deviation from the preset position or not. The information indicating the position of the particle beam P detected by the position monitor 8 is inputted to the control computer 11.

The ridge filter 9 is installed to widen the Bragg peak of the dose in the depth direction inside the patient's body.

The range shifter 10 controls the irradiation position of the lesion site T in the Z-axis direction. The range shifter 10 is composed of a plurality of acrylic plates, which are different in thickness from each other, for example. When these acrylic plates are combined, the energy of the particle beam P passing through the range shifter 10, i.e., the range within the body, can be stepwisely changed. The range shifter 10 can generate the Bragg peak at a preset position of the lesion site T in the Z-axis direction. The range shifter 10 is controlled by the control computer 11.

Instead of the range shifter 10, the range within the body can also be changed by changing the energy of the particle beam P at the time of being emitted from the beam accelerator 3.

The control computer 11 controls the entirety of the particle beam control system 1. For example, the control computer 11 measures the irradiation dose for each grid point of the lesion site T, checks normality of the irradiation position for each spot, and controls on/off of beam emission regarding to the beam accelerator 3. Furthermore, the control computer 11 outputs an instruction regarding scanning to the beam scanner 4 and controls the combination of acrylic plates for the range shifter 10, for example.

Next, the control aspect of the beam scanner 4 and the scanning electromagnets 5 and 6 by the control computer 11 will be described by referring to FIG. 2. In FIG. 2, illustrations of components excluding the beam scanner 4, the scanning electromagnets 5 and 6, the position monitor 8, and the control computer 11 are omitted.

The beam scanner 4 supplies powers to the respective scanning electromagnets 5 and 6. For example, the beam scanner 4 includes an X-axis power supply 12 configured to supply powers to the pair of X-axis electromagnets 5A and 5B and a Y-axis power supply 13 configured to supply powers to the pair of Y-axis electromagnets 6A and 6B.

The X-axis electromagnets 5A and 5B adjust the trajectory of charged particles, which are made incident on the generated magnetic field, in the X-axis direction (i.e., in the horizontal direction). The Y-axis electromagnets 6A and 6B adjust the trajectory of charged particles, which are made incident on the generated magnetic field, in the Y-axis direction (i.e., in the vertical direction).

The control computer 11 includes an input device 14, an output device 15, a communication device 16, a controller 17, and a memory 18.

The control computer 11 includes hardware resources such as a Central Processing Unit (CPU), a Graphics Processing Unit (GPU), a Read Only Memory (ROM), a Random Access Memory (RAM), a Hard Disk Drive (HDD) and/or a Solid State Drive (SSD), and is configured as a computer in which information processing by software is achieved with the use of the hardware resources by causing the CPU to execute various programs. Further, the particle beam control method of the present embodiment is achieved by causing the computer to execute the various programs.

Moreover, each configuration of the control computer 11 does not necessarily have to be provided in one computer. For example, the configuration of the control computer 11 may be achieved by a plurality of computers that are interconnected via a network.

Predetermined information is inputted to the input device 14 in response to the operation by a user who uses the control computer 11. The input device 14 includes an input interface such as a mouse, a keyboard, and a touch panel. That is, the predetermined information is inputted to the input device 14 depending on the operation on these input interface.

The output device 15 outputs the predetermined information. The control computer 11 includes an interface that displays an image such as a display that outputs analysis results. This output device 15 controls images to be displayed on the display. The display may be separated from the main body of the computer or may be integrated with the main body of the computer.

Additionally, or alternatively, the control computer 11 may control images to be displayed on the display of other computers interconnected via the network. In that case, the output device 15 included in the other computers may control the output of specified information.

The communication device 16 communicates with other computers via a communication line. For example, the control computer 11 and other computers may be interconnected via a LAN (Local Area Network), a WAN (Wide Area Network), a mobile communication network, or the Internet.

The controller 17 controls the scanning electromagnets 5 and 6. The controller 17 has a centroid-position calculation function 19 and a correction-value calculation function 20. These are implemented by causing it's the CPU to execute the programs stored in the memory or the HDD.

The memory 18 stores various information items necessary for the controller 17 to control the scanning electromagnets 5 and 6. The memory 18 has a magnet-information storage function 21 and a correction-value storage function 22.

For example, the memory 18 cumulatively stores dose profile of the particle beam P. This dose profile is sent to the output device 15 in units of slices. The dose profile in units of slices is then displayed on the display screen in a manner visible to the user.

The controller 17 sets current values of electric currents to be supplied to the respective scanning electromagnets 5 and 6. For example, the controller 17 sets the current values of the powers to be outputted by the respective X-axis power supply 12 and Y-axis power supply 13 in accordance with the preset irradiation pattern. The X-axis power supply 12 outputs the excitation currents based on the irradiation pattern to the respective X-axis electromagnets 5A and 5B, and the Y-axis power supply 13 outputs the excitation currents based on the irradiation pattern to the respective Y-axis electromagnets 6A and 6B.

The controller 17 sets irradiation information (X, Y, Ix, Iy, En) of the spot position based on the design theory. In this irradiation information, X and Y are the spot coordinates (position) for the theoretical particle trajectory, Ix and Iy are the respective current setting values of the X-axis power supply 12 and the Y-axis power supply 13, and En is a beam energy value.

The controller 17 controls the respective current values of the powers to be outputted by the X-axis power supply 12 and the Y-axis power supply 13 on the basis of the current setting values of irradiation information. The particle beam P is deflected in the X-axis direction and in the Y-axis direction by controlling these current values, and thereby, the lesion site T is scanned two-dimensionally.

The position monitor 8 detects the position of the particle beam P when the particle beam P is deflected two-dimensionally. This position is expressed by the coordinates of the X-axis direction and the Y-axis direction, which are orthogonal to each other.

On the basis of the position of the particle beam P detected (i.e., actually measured) by the position monitor 8, the centroid-position calculation function 19 calculates the centroid position, which is the actual irradiation position of the particle beam P. This calculated centroid position is inputted to the correction-value calculation function 20.

The correction-value calculation function 20 calculates deviation amount between the centroid position and the spot position, which is the designed irradiation position of the scanning electromagnets 5 and 6. On the basis of the deviation amount between the centroid position and the spot position, the correction-value calculation function 20 calculates at least one correction value for correcting the centroid position to the spot position. For example, the correction-value calculation function 20 calculates two correction values for the two current setting values (Ix, Iy) of the irradiation information. The centroid position is corrected or adjusted so as to match the spot position on the basis of these correction values.

Note that each correction value to be calculated differs depending on the ion species and the beam energy value (En) to be used for the particle beam P. For example, when the ion species of the particle beam P is switched, a correction value is calculated depending on the beam energy value of this ion species.

For example, it is assumed that the spot position (X,Y) in the theoretical particle trajectory corresponding to the two current setting values (Ix, Iy) is set. Under this assumption, it is also assumed that the actual spot position (i.e., the centroid position) calculated by the centroid-position calculation function 19 deviates from the ideal spot position and becomes (X′, Y′). In this case, a function of two current values, which is (I′x (X, Y, X′, Y′, Ix, Iy, En), I′y (X, Y, X′, Y′, Ix, Iy, En)), is calculated. On the basis of this function, two correction values for correcting the two current values are calculated from the difference in deviation amount between the theoretical spot position and the actual spot position (i.e., the centroid position) such that the theoretical spot position (X,Y) is achieved. These two correction values are stored in the correction-value storage function 22.

In other words, the correction-value storage function 22 preliminarily or previously stores the correction values before start of the particle beam treatment using the particle beam P. In this configuration, the actual irradiation position of the particle beam P can be adjusted in advance, and the particle beam P can be radiated accurately from the beginning of the particle beam treatment.

In addition, the correction-value storage function 22 stores the correction information indicating the correction values calculated by the correction-value calculation function 20 in association with the ion species and the beam energy values.

For example, the correction-value storage function 22 stores either or both a plurality of correction values corresponding to respective ion species to be used in the particle beam P and a plurality of beam energy values corresponding to respective beam energy values to be used in the particle beam P. On the basis of either or both the ion species and the beam energy values to be used at the time of the particle beam treatment, the controller 17 selects correction values corresponding to either or both. In this configuration, the correction values can be switched depending on the state of the particle beam P, such as the ion species and the beam energy value, and thus, the particle beam P can be adjusted appropriately.

The magnet-information storage function 21 stores magnet information indicating the actual arrangement of the scanning electromagnets 5 and 6. For example, the magnet-information storage function 21 stores the spot position of the X-axis electromagnets 5A and 5B and the Y-axis electromagnets 6A and 6B in the particle trajectory based on the design theory. The spot position at this time can form or achieve an arbitrary irradiation shape. For example, when there is an irradiation pattern with a square irradiation shape, the magnet-information storage function 21 stores the spot position that serves as a reference for making the irradiation shape square. Corresponding to this reference spot position, a reference current value is also stored. The magnet-information storage function 21 also stores the spot position unique to the arrangement aspect of the X-axis electromagnets 5A and 5B and the Y-axis electromagnets 6A and 6B.

In addition, the irradiation pattern is a pattern obtained by adding correction values to the irradiation information (i.e., current setting values) of the centroid position that is calculated by the correction-value calculation function 20.

Furthermore, during construction or periodic maintenance of the particle beam control system 1, the spot position of the test-radiated particle beam P is shifted or deviated in some cases. In this case, the correction information stored in the correction-value storage function 22 may be updated with the correction values (correction information) that are newly calculated by the correction-value calculation function 20.

For example, positioning (i.e., alignment) of the two scanning electromagnets 5 and 6 is performed before start of the particle beam treatment using the particle beam P, and if the spot position is deviated during this positioning, the correction values are calculated before start of the particle beam treatment. In this configuration, at the time of positioning of the scanning electromagnets 5 and 6, the correction values already stored in the correction-value storage function 22 can be updated.

During the particle beam treatment, the irradiation information having been set in the treatment plan is corrected by using the correction information stored in the correction-value storage function 22. For example, the controller 17 controls the current values of the powers to be supplied to the respective scanning electromagnets 5 and 6 on the basis of the corrected irradiation information. In this configuration, even if the installation positions of the scanning electromagnets 5 and 6 are deviated, the current values can be automatically corrected. Hence, the particle beam P is radiated at the assumed irradiation position. Consequently, the irradiation position of the particle beam P can be constantly corrected, or the time required for positioning of the scanning electromagnets 5 and 6 can be reduced.

In this configuration, even if it is difficult to disassemble the scanning electromagnets 5 and 6, the irradiation position of the particle beam P can be adjusted.

In addition, the control computer 11 can display the spot position related to magnetic field correction. In the present embodiment, the display (i.e., the output device 15) of the control computer 11 constitutes a spot position display.

For example, the irradiation information indicating the deviation between the centroid position and the theoretical spot position stored in the magnet-information storage function 21 can be displayed on the display. The user can check the theoretical spot position and the corrected centroid position. Aside from these information items, the beam energy value, the ion species, and the deviation amount after correction can also be displayed on the display.

Note that “the deviation amount” in the present embodiment includes information such as rotation and distortion of the magnetic fields to be generated by the scanning electromagnets 5 and 6. In addition, correction of the deviation amount is to correct the current values of the scanning electromagnets 5 and 6, which have been set during treatment planning, during the actual particle beam treatment.

The user checks the spot position that is based on the design theory and serves as a reference. On the basis of the irradiation information based on the design theory, the user similarly checks the spot position and the centroid position that is the actual spot position after correction. In this configuration, before irradiation to the patient, the user can check whether the positions of the scanning electromagnets 5 and 6 are deviated from the ideal positions or not. In addition, the particle beam P can be radiated to the assumed irradiation position by checking the corrected irradiation information in advance. Moreover, the position of the particle beam P can be constantly corrected. Furthermore, the time required for positioning of the scanning electromagnets 5 and 6 can be reduced.

The control computer 11 displays information indicating at least one of the centroid position before or after correction, the spot position, the deviation amount, and the correction value on the screen of the display (i.e., the output device 15). In this configuration, the user can check the deviation amount of the centroid position, for example. The ion species and/or the beam energy value may also be displayed, for example.

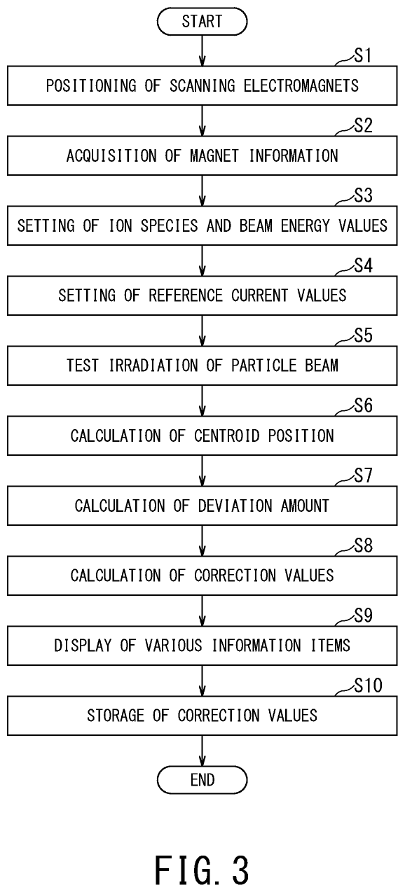

Next, the correction processing of the particle beam P will be described on the basis of the flowchart of FIG. 3 by referring to the above-described other figures as required. The following steps are at least some of the processing included in the correction processing, and other steps may also be included in the correction processing.

In the first step S1, before start of the particle beam treatment using the particle beam P, for example, during construction or periodic maintenance of the particle beam control system 1, positioning of the two scanning electromagnets 5 and 6 is performed. The user (i.e., worker) performs the positioning of the two scanning electromagnets 5 and 6.

In the next step S2, on the basis of information entered by the user into the input device 14 or information received by the communication device 16, the controller 17 acquires the magnet information indicative of the actual arrangement aspect of the scanning electromagnets 5 and 6. This magnet information is stored in the magnet-information storage function 21. The magnet information includes the design information of the scanning electromagnets 5 and 6.

In the next step S3, on the basis of the information entered by the user into the input device 14 or the information received by the communication device 16, the controller 17 sets the ion species and the beam energy value to be used in the particle beam P.

In the next step S4, on the basis of the information entered by the user into the input device 14 or the information received by the communication device 16, the controller 17 sets the reference current values, which are design references at the time of supplying powers from the beam scanner 4 to the scanning electromagnets 5 and 6. For example, an X-axis reference current value at the time of supplying powers from the X-axis power supply 12 to the X-axis electromagnets 5A and 5B is set, and a Y-axis reference current value at the time of supplying powers from the Y-axis power supply 13 to the Y-axis electromagnets 6A and 6B is set.

In the next step S5, the controller 17 controls the beam generator 2, the beam accelerator 3, the beam scanner 4, and the scanning electromagnets 5 and 6 so as to perform test irradiation of the particle beam P. The position monitor 8 detects the irradiation position of the particle beam P in the X-axis direction and the Y-axis direction. This information indicating the irradiation position of the particle beam P is inputted to the controller 17.

In the next step S6, on the basis of the position of the particle beam P detected by the position monitor 8, the centroid-position calculation function 19 calculates the centroid position that is the actual irradiation position of the particle beam P.

In the next step S7, the correction-value calculation function 20 calculates the deviation amount between the centroid position and the spot position, which is the designed irradiation position of the scanning electromagnets 5 and 6.

In the next step S8, the correction-value calculation function 20 calculates the correction values for correcting the centroid position to the spot position on the basis of the deviation amount between the spot position and the centroid position. Note that the correction values include an X-axis correction value for correcting the X-axis reference current value and a Y-axis correction value for correcting the Y-axis reference current value.

In the next step S9, the controller 17 displays the information indicating at least one of the centroid position before or after correction, the spot position, the deviation amount, and the correction values on the screen of the display (i.e., the output device 15).

In the next step S10, the controller 17 stores the calculated correction values in the correction-value storage function 22, and then the correction processing is completed.

If there are a plurality of ion species and a plurality of beam energy values, the processing from the step S3 to the step S10 is repeated by changing the ion species and the beam energy value each time of repetition. In other words, a plurality of correction values corresponding to respective combinations of the ion species and the beam energy value are stored in the correction-value storage function 22.

When the deviation amount between the spot position and the centroid position is large, the user may perform positioning of the scanning electromagnets 5 and 6 again. In this case, the processing from the step S1 is restarted.

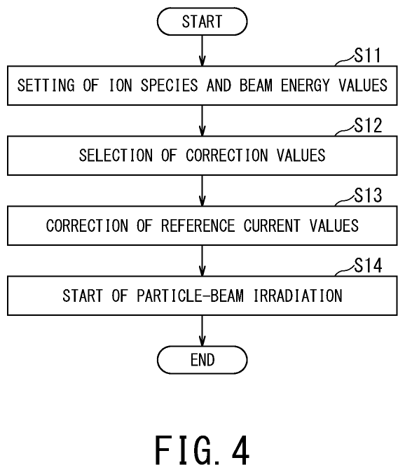

Next, irradiation start processing of the particle beam P will be described on the basis of the flowchart of FIG. 4 by referring to the above-described other figures as required. The following steps are at least some of the processing included in the irradiation start processing, and other steps may also be included in the irradiation start processing.

In the first step S11, when the particle beam treatment using the particle beam P is started, the controller 17 sets the ion species and the beam energy values to be used in the particle beam P on the basis of the information entered by the user into the input device 14 or the information received by the communication device 16.

In the next step S12, the controller 17 reads out the correction values stored in the correction-value storage function 22. For example, the controller 17 selects the correction values corresponding to the preset ion species and beam energy value from among the plurality of correction values stored in the correction-value storage function 22.

In the next step S13, the controller 17 corrects the reference current values on the basis of the correction values stored in the correction-value storage function 22. For example, the controller 17 corrects the X-axis reference current value by using the X-axis correction value, and also corrects the Y-axis reference current value by using the Y-axis correction value.

In the next step S14, the controller 17 controls the beam generator 2, the beam accelerator 3, the beam scanner 4, and the scanning electromagnets 5 and 6 so as to start irradiation of the particle beam P, and then, the irradiation start processing is completed.

Even during the particle beam treatment, the deviation amount between the spot position and the centroid position may be calculated so that new correction values are calculated, and the existing correction values are updated.

Although a mode in which each step is executed in series is illustrated in the above flowcharts, the execution order of the respective steps is not necessarily fixed and the execution order of part of the steps may be changed. Additionally, some steps may be executed in parallel with another step.

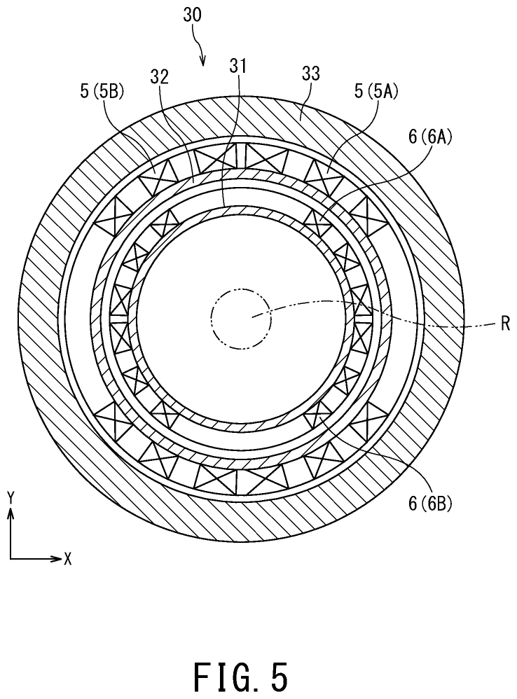

Next, the first modification will be described by using FIG. 5 to FIG. 7. The same components as the above-described components are denoted by the same reference signs, and duplicate descriptions are omitted.

As shown in FIG. 5, the particle beam control system 1 (FIG. 1) according to the first modification includes an electromagnetic structure 30. The electromagnetic structure 30 includes the scanning electromagnets 5 and 6, a first cylindrical member 31, a second cylindrical member 32, and a third cylindrical member 33. The electromagnetic structure 30 constitutes part of the transport path of the particle beam P (FIG. 1) in the particle beam control system 1.

Each of the first to third cylindrical members 31, 32, 33 has a cylindrical shape (i.e., hollow shape) and is a member, inner diameter (i.e., aperture) of which is constant along the cylindrical axis C. When the first to third cylindrical members 31, 32, 33 extend in a straight line, the cylindrical axis C and the Z-axis direction are the same. The first to third cylindrical members 31, 32, 33 are arranged concentrically (i.e., coaxially) around a passage region R through which the particle beam P passes.

The first cylindrical member 31 is a vacuum duct, inside of which is vacuumized. The Y-axis electromagnets 6A and 6B are arranged on the outer circumferential surface of the first cylindrical member 31.

The second cylindrical member 32 is provided so as to cover the outside of the first cylindrical member 31. The X-axis electromagnets 5A and 5B are arranged on the outer circumferential surface of the second cylindrical member 32.

The third cylindrical member 33 is provided so as to cover the outside of the second cylindrical member 32. The third cylindrical member 33 serves as a cover that constitutes the outer periphery of the electromagnetic structure 30.

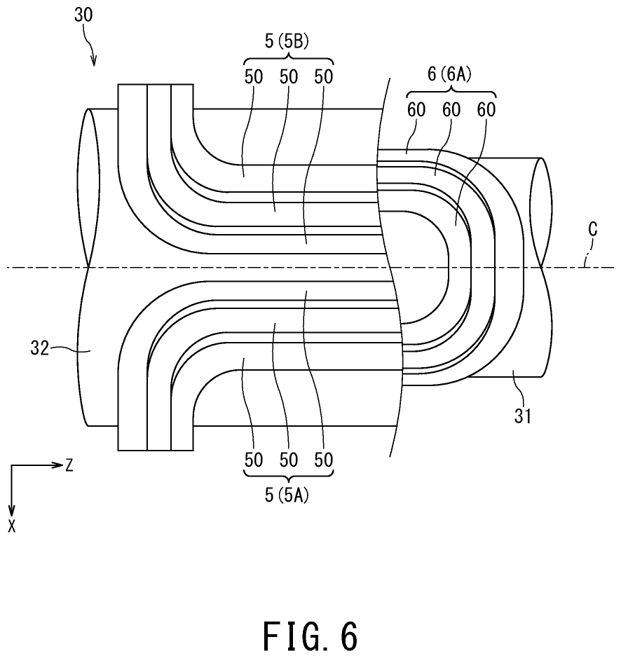

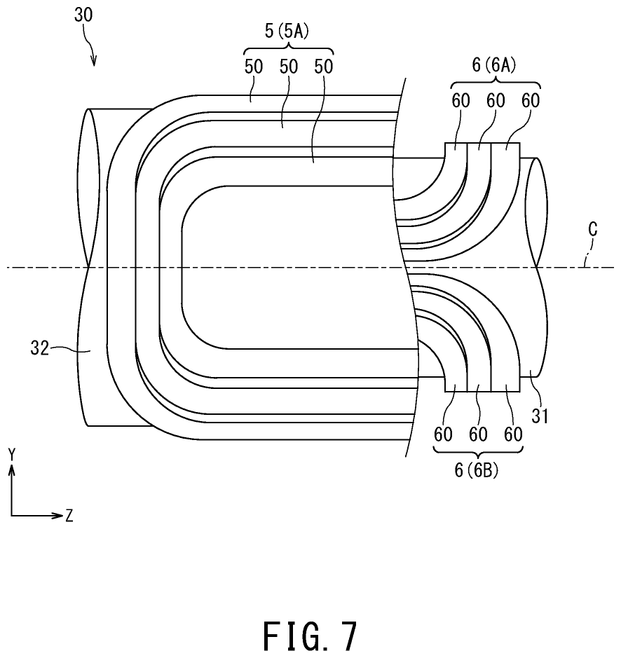

As shown in FIG. 6 and FIG. 7, each of the pair of X-axis electromagnets 5A and 5B is composed of a plurality of coils 50. Similarly, each of the pair of Y-axis electromagnets 6A and 6B is composed of a plurality of coils 60. Furthermore, in FIG. 6 and FIG. 7, the illustration of the third cylindrical member 33 is omitted in order to avoid complication and facilitate understanding.

In the first modification, the X-axis electromagnets 5A and 5B and the Y-axis electromagnets 6A and 6B are arranged concentrically (i.e., coaxially) and partially overlap each other in the circumferential direction (i.e., in the X-axis direction and the Y-axis direction). In this configuration, the X-axis electromagnets 5A and 5B and the Y-axis electromagnets 6A and 6B can be disposed together compactly.

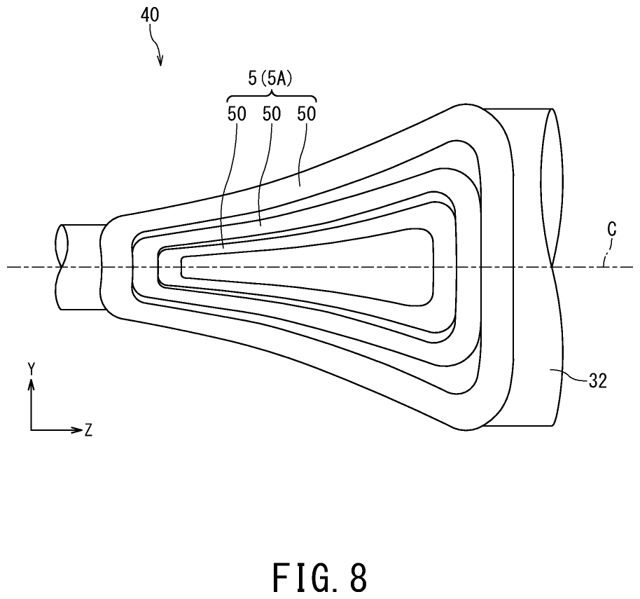

Next, the second modification will be described by using FIG. 8. The same components as the above-described components are denoted by the same reference signs, and duplicate descriptions are omitted.

An electromagnetic structure 40 of the second modification has a shape in which its inner diameter (i.e., diameter) increases continuously along the traveling direction of the particle beam P (i.e., along the axis C). In other words, this electromagnetic structure 40 has a shape in which its diameter increases from the incident side (i.e., upstream side) toward the emission side (i.e., downstream side) of the particle beam P.

In the aspect of FIG. 8, the shape of the electromagnetic structure 40 in which the inner diameter expands in the above-described manner is a shape that matches the deflection of the particle beam P, and is the same shape as a tip of a trumpet of a musical instrument. As to the inner diameter of the electromagnetic structure 40, the electromagnetic structure 40 may have a shape in which the inner diameter increases linearly from the incident side to the emission side of the particle beam P.

As to the inner diameter (aperture) of each of the first to third cylindrical members 31, 32, 33 (FIG. 5 to FIG. 7), each of these members 31, 32, 33 also has a shape in which the inner diameter increases continuously along the traveling direction of the particle beam P.

The X-axis electromagnets 5A and 5B provided on the outer circumferential surface of the second cylindrical member 32 are shaped to expand as the diameter of the second cylindrical member 32 becomes larger. The Y-axis electromagnets 6A and 6B provided on the outer circumferential surface of the first cylindrical member 31 are also shaped to expand as the diameter of the first cylindrical member 31 becomes larger.

In the second modification, the inner diameter of the electromagnetic structure 40 is reduced on the incident side of the electromagnetic structure 40, wherein this incident side is a stage before the particle beam P is scanned. On the emission side where the particle beam P is expanded after being scanned, the inner diameter of the electromagnetic structure 40 is increased along the beam trajectory. In this configuration, the coils 50 and 60 (FIG. 5 to FIG. 7) can be brought close to the particle beam P without causing the particle beam P to collide with the inner circumferential surface of the electromagnetic structure 40, and a wide irradiation field of the particle beam P can be secured. In other words, even if the amplitude of the deflection of the particle beam P is increased, the particle beam P can be prevented from interfering with the electromagnetic structure 40.

It may configured such that the X-axis electromagnets 5A and 5B and the Y-axis electromagnets 6A and 6B as a whole constitute at least one electromagnet unit (not shown) and these electromagnet units are arranged side by side in the Z-axis direction. Furthermore, each electromagnet unit may be configured to increase in inner diameter along the traveling direction of the particle beam. This configuration can widen the irradiation field of the particle beam P while suppressing increase in size of the entire system composed of the plurality of electromagnet units (i.e., increase in aperture over the entire length).

The above control computer 11 includes a control device, a storage device, an output device, an input device, and a communication interface. The control device includes a highly integrated processor such as a Central Processing Unit (CPU), a Graphics Processing Unit (GPU), a Field Programmable Gate Array (FPGA), and a special-purpose chip. The storage device includes a Read Only Memory (ROM), a Random Access Memory (RAM), a Hard Disk Drive (HDD), a Solid State Drive (SSD), or the like. The output device includes a display panel, a head-mounted display, a projector, a printer, or the like. The input device includes a mouse, a keyboard, a touch panel, or the like. The control computer 11 can be achieved by hardware configuration with the use of the normal computer.

Note that the program executed in the above control computer 11 is provided by being incorporated in a memory such as the ROM in advance. Additionally, or alternatively, the program may be provided by being stored as a file of installable or executable format in a non-transitory computer-readable storage medium such as a CD-ROM, a CD-R, a memory card, a DVD, and a flexible disk (FD).

In addition, the program executed in the control computer 11 may be stored on a computer connected to a network such as the Internet and be provided by being downloaded via a network. Further, the control computer 11 can also be configured by interconnecting and combining separate modules, which independently exhibit respective functions of the components, via a network or a dedicated line.

According to the above-described embodiments, the task of adjusting the deviation in the irradiation position of the particle beam P can be simplified by correcting the current values that are the reference values in design at the time of supplying powers from the X-axis power supply 12 and the Y-axis power supply 13 to the scanning electromagnets 5 and 6.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions. The articles “the”, “a” and “an” are not necessarily limited to mean only one, but rather are inclusive and open ended so as to include, optionally, multiple such elements.

1. A particle beam control system comprising:
two scanning electromagnets configured to scan a particle beam in two-dimensional directions and be different in direction of deflecting the particle beam from each other;
two power supplies configured to supply respective powers to the two scanning electromagnets;
a position monitor configured to detect a position of the particle beam; and
a control computer configured to control the two scanning electromagnets,
wherein the control computer is configured to:
calculate a centroid position by using the position of the particle beam detected by the position monitor, the centroid position being an actual irradiation position of the particle beam;
calculate deviation amount between the centroid position and a spot position that is a designed irradiation position of the two scanning electromagnets;
calculate at least one correction value for correcting the centroid position to the spot position by using the deviation amount;
store the at least one correction value in a memory; and
correct at least one current value by using the at least one correction value stored in the memory, the at least one current value being a design reference when power is supplied from at least one of the two power supplies to at least one of the two scanning electromagnets.

2. The particle beam control system according to claim 1, wherein the memory is configured to preliminarily store the at least one correction value before start of particle beam treatment using the particle beam.

3. The particle beam control system according to claim 1, wherein:
the two scanning electromagnets include an X-axis electromagnet configured to deflect the particle beam in an X-axis direction and a Y-axis electromagnet configured to deflect the particle beam in a Y-axis direction; and
the X-axis electromagnet and the Y-axis electromagnet are provided at a same position in a Z-axis direction that is a traveling direction of the particle beam.

4. The particle beam control system according to claim 3, wherein the X-axis electromagnet and the Y-axis electromagnet are arranged concentrically and partially overlap each other in a circumferential direction.

5. The particle beam control system according to claim 3, wherein:
the X-axis electromagnet and the Y-axis electromagnet constitute at least one electromagnet unit; and
the at least one electromagnet unit has a shape in which an inner diameter increases along the traveling direction.

6. The particle beam control system according to claim 1, wherein:
the at least one correction value comprises a plurality of correction values;
the memory is configured to store either or both the plurality of correction values corresponding to respective ion species to be used in the particle beam and the plurality of correction values corresponding to respective beam energy values to be used in the particle beam;
the control computer is configured to select the at least one correction value corresponding to at least one of two information items from the plurality of correction values,
one of the two information items being at least one ion species among the plurality of ion species to be used for particle beam treatment,
another of the two information items being at least one beam energy value among the plurality of beam energy values to be used for the particle beam treatment.

7. The particle beam control system according to claim 1, wherein the at least one correction value is calculated in accordance with positioning before start of particle beam treatment when the positioning of the two scanning electromagnets is performed before the start of particle beam treatment using the particle beam.

8. The particle beam control system according to claim 1, wherein the control computer is configured to display information indicating at least one of the centroid position before or after correction, the spot position, the deviation amount, and the at least one correction value.

9. A particle beam control method that uses:
two scanning electromagnets configured to scan a particle beam in two-dimensional directions and be different in direction of deflecting the particle beam from each other;
two power supplies configured to supply respective powers to the two scanning electromagnets;
a position monitor configured to detect a position of the particle beam; and
a control computer configured to control the two scanning electromagnets,
the particle beam control method comprising steps of:
calculate a centroid position by using the position of the particle beam detected by the position monitor, the centroid position being an actual irradiation position of the particle beam;
calculate deviation amount between the centroid position and a spot position that is a designed irradiation position of the two scanning electromagnets;
calculate at least one correction value for correcting the centroid position to the spot position by using the deviation amount;
store the at least one correction value in a memory; and
correct at least one current value by using the at least one correction value stored in the memory, the at least one current value being a design reference when power is supplied from at least one of the two power supplies to at least one of the two scanning electromagnets.
